(19) United States
(12) Patent Application Publication
(10) Pub. No.: US 2024/0398485 A1
(43) Pub. Date: Dec. 5, 2024

(54) CHIP-LEVEL POSITIONING METHOD FOR ORTHOPEDIC SURGERY NAVIGATION BASED ON ULTRA-WIDE BANDWIDTH

(71) Applicant: JILIN UNIVERSITY, Changchun City (CN)

(72) Inventors: Fei YIN, Changchun City (CN); Shijie LV, Changchun City (CN); Yijun WANG, Changchun City (CN); Jinquan WANG, Changchun City (CN)

(21) Appl. No.: 18/622,962

(22) Filed: Mar. 31, 2024

(30) Foreign Application Priority Data
May 22, 2023 (CN) 202310578202.3

(51) Int. Cl. A61B 34/20; G01S 11/02

(52) U.S. Cl. CPC A61B 34/20; G01S 11/02; A61B 2034/2051; A61B 2034/2059

(57) ABSTRACT

Provided is a chip-level positioning method for orthopedic surgery navigation based on an Ultra-wide Bandwidth (UWB). A UWB chip-level wireless positioning module is installed on a surgical instrument. The biggest advantage of the UWB wireless positioning module is that the UWB wireless positioning module has a wide bandwidth, so it has good anti-interference ability. In a positioning and tracking stage, only a base station and modules need to communicate to acquire spatial position of the surgical instruments relative to patients. The wireless positioning technology based on the UWB is very mature at this stage, and there are diverse intraoperative visualization means, and surgical status can be monitored through devices such as mobile phones and tablets. Compared with an existing optical positioning system, the present disclosure has the advantages of good robustness, high cost performance, strong real-time performance and the like.

### CROSS-REFERENCE TO RELATED APPLICATION

This patent application claims the benefit and priority of Chinese Patent Application No. 202310578202.3 filed with the China National Intellectual Property Administration on May 22, 2023, the disclosure of which is incorporated by reference herein in its entirety as part of the present application.

### TECHNICAL FIELD

The present disclosure relates to the technical field of surgical navigation and positioning, in particular to a chip-level positioning method for orthopedic surgery navigation based on an Ultra-wide Bandwidth (UWB).

### BACKGROUND

The spatial positioning technology of surgical navigation is responsible for calculating the spatial position and posture of a patient entity and a surgical instrument, and realizing real-time monitoring and control during the surgical process by positioning a positioning device at the end of the surgical instrument and substituting a spatial position and posture information collected by the positioning device into an established spatial transformation relationship, where the established spatial conversion relationship is a mapping relationship between a surgical instrument coordinate system, a patient coordinate system and a robot coordinate system (i.e., a coordinate system of the positioning device). In a surgical navigation system, the transformation relationship among a surgical instrument coordinate system, a patient coordinate system and a robot coordinate system can be established by the spatial positioning technology. At present, positioning can be classified into electromagnetic positioning, ultrasonic positioning and optical positioning according to different positioning principles.

The hardware of an electromagnetic positioning method includes a detector and a transmitter of a magnetic field, generally including 3 transmitters and 1 detector. When the transmitter is placed under the operating table, the detector receives signals from the transmitters and the position of the detector can be determined based on received signals and a relative position between the detector and the transmitters. By detecting the direction and the intensity of the magnetic field in the surgical area, the position of the target is calculated, with a positioning error of 1-3 mm.

The ultrasonic positioning method is to fix a receiver on the surgical instrument, calculate the distance from the receiver to a transmitter through a propagation time and a sound velocity of a sound wave, and then determine the position of the receiver by expanding search radius, in which an error is 4-5 mm generally.

The optical positioning method can be classified into an infrared optical positioning method and a visible optical positioning method. The infrared optical positioning method can be classified into a target active optical positioning method and a camera active optical positioning method. The optical positioning system requires that a positioning bracket is installed on the body surface mapping position of a lesion site and on the surgical instrument, respectively. The bracket is provided with some feature points, such as infrared light emitting diodes, fluorescent balls reflecting infrared light, black and white checkerboards, etc. The spatial information of any rigid body can be theoretically determined by three non-collinear spatial points thereon. The optical positioning method is to locate the feature points on the bracket, so that the information about the distance and the angle between the surgical instrument and the lesion site can be calculated.

The disadvantage of the electromagnetic positioning method is that it is very sensitive to metal objects and susceptible to interference, especially ferromagnetic instruments like high-frequency scalpels, which have a great influence on the accuracy of electromagnetic positioning.

The initial setting of the ultrasonic positioning method is based on an ideal sound velocity, but a big error can occur when the environment changes, which will affect the positioning accuracy of the surgical localization.

The optical positioning method will affect the positioning accuracy when the light is blocked, so that it is necessary to increase the number of cameras to improve the reliability of the positioning method. Moreover, the current mainstream optical positioning system is very cumbersome in preoperative configuration of the surgical environment and the system operation is complicated, so that there is a big deviation between the doctor's mastery of the device and the current utilization rate. The device is expensive, and the cost performance is not high.

### SUMMARY

The present disclosure provides a method for real-time positioning and tracking of surgical instruments based on the UWB chip-level positioning technology. UWB positioning has the advantages of high resolution, wide frequency spectrum, low power consumption and strong penetration. The method has the advantages of convenient operation, high cost performance, high accuracy, strong real-time performance and high robustness when positioning and tracking surgical instruments.

In order to achieve the above purpose, the present disclosure provides the following technical scheme: a chip-level positioning method for orthopedic surgery navigation based on a UWB, including:
S1, laying a positioning base station and arranging a UWB wireless positioning module on a surgical instrument, where the positioning base station and the UWB wireless positioning module arranged on the surgical instrument use a same UWB module unit;
S2, real-time positioning: first, substituting an original Time Difference of Arrival (TDOA) value into a Chan algorithm to calculate and acquire a preliminary positioning coordinate of a UWB positioning tag, thereafter, calculating a residual sum of squares to set a threshold, where the residual sum of squares is used to deal with positioning errors in the UWB wireless positioning module, and eliminating TDOA measurement values with errors higher than the threshold, and substituting the screened TDOA measurement values that meet requirements back into a standard Extended Kalman Filter (EKF) filtering algorithm, so as to obtain a final position coordinate of the surgical instrument.

In some embodiments, specific Step S2 includes:
S21, TDOA positioning;
by using a TDOA algorithm, measuring a time difference between moments of two reference base stations receiving broadcast signals sent by a target node, thus calculating a distance difference between distances from the target node to the two reference base stations by multiplying a wave velocity by the time difference, obtaining a hyperbolic equation by taking the two reference base stations as focuses of a curve and taking the distance difference as 2a, where one hyperbolic equation is incapable of determining the target node, and at least three base stations are used to solve two hyperbolic equations, and an intersection point of two hyperbolas determined by the two hyperbolic equations is a target node;
S22, the Chan algorithm for positioning based on TDOA technology;
using a two-step Weighted Least Square (WLS) method to locate and calculate a target position, which is suitable for both small-scale and large-scale positioning systems, where during the calculating, a nonlinear TDOA equations are first processed and converted into linear equations, and then an initial solution is estimated through WLS; and thereafter, the initial solution is calculated through the WLS for a second time to further estimate coordinate of the positioning tag, where equation conditions used in the first WLS calculation, such as constraints, are the same as those used in the second WLS calculation;
S23, the EKF algorithm for positioning based on TDOA technology;
in a UWB positioning system based on TDOA technology, considering that the TDOA equations are nonlinear equations, using an EKF to solve a nonlinear problem, where the idea of the EKF is that for a nonlinear system, the system is discretized by means of numerical analysis, and Taylor expansion is carried out in a neighborhood of calculation points, terms above quadratic terms are deleted, and only primary terms are reserved, and thus the Kalman filter is applied to the nonlinear system;
S24, estimation of the position coordinate of the positioning tag;
substituting screened TDOA measurement values with small errors into the standard EKF filtering algorithm, so as to obtain a final position coordinate of the surgical instrument.

In some embodiments, a specific linear equation of TDOA positioning in Step S21 is as follows:
coordinates of base stations are set to (x₁, y₁), (x₂, y₂) and (x₃, y₃) clockwise from the BS₁, and a tag coordinate to be solved is (x, y), the time for a measure signal traveling from a node to be tested to BS₁ is tᵢ (i=2,3), and BS₁ and BSᵢ are deemed as focuses, a hyperbolic equation is drawn with D_{i,1}=dᵢ−d₁=2a, and a distance relationship between the node to be tested and the base station i is obtained by a distance formula between two points:

$${D}_{i} = \sqrt{{( {x}_{i} - x )}^{2}} - \sqrt{{( {y}_{i} - y )}^{2}}$$

a relationship between a distance difference between the distance from the node to be tested to a main positioning base station BS₁ and the distance Dᵢ from the node to be tested to other base stations and the time difference is as follows:

$${D}_{i , 1} = {D}_{i} - {D}_{1} = {ct}_{i , 1} = \sqrt{{( {x}_{i} - x )}^{2} + {( {y}_{i} - y )}^{2}} - \sqrt{{( {x}_{1} - x )}^{2} + {( {y}_{1} - y )}^{2}}$$

c indicates a propagation velocity of an electromagnetic wave emitted by the target in the medium, and more than two base stations besides the main base station are needed to complete the determination of the target node, from which following hyperbolic nonlinear equations are obtained:

$$\{ \begin{matrix}{D}_{2 , 1} = {D}_{2} - {D}_{1} = \sqrt{{( {x}_{2} - x )}^{2} + {( {y}_{2} - y )}^{2}} - \sqrt{{( {x}_{1} - x )}^{2} + {( {y}_{1} - y )}^{2}} \\ {D}_{3 , 1} = {D}_{3} - {D}_{1} = \sqrt{{( {x}_{3} - x )}^{2} + {( {y}_{3} - y )}^{2}} - \sqrt{{( {x}_{1} - x )}^{2} + {( {y}_{1} - y )}^{2}}\end{matrix}$$

x and y are solved using the Chan algorithm to obtain the tag coordinate.

In some embodiments, a specific linear equation of the EKF algorithm based on TDOA technology positioning in Step S23 is as follows:
assuming that the nonlinear system is:

$$\{ \begin{matrix}{x}_{k} = f ( {x}_{k - 1} , {u}_{k - 1} , {w}_{k - 1} ) \\ {z}_{k} = h ( {x}_{k} , {v}_{k} )\end{matrix}$$

a time update equation is:

$$\begin{matrix}{\hat{x}}_{k}^{-} = f ( {\hat{x}}_{k - 1} , {u}_{k - 1} , {w}_{k - 1} ) \\ {P}_{k}^{-} = ϕ {P}_{k - 1} {ϕ}^{T} + Q\end{matrix}$$

an updated measurement equation is:

$$\begin{matrix}{K}_{k} = {P}_{k}^{-} {{H}_{K}^{T} ( {H}_{k} {P}_{k}^{-} {H}_{K}^{T} + R )}^{- 1} \\ {\hat{x}}_{k} = {\hat{x}}_{k - 1}^{-} + {K}_{k} ( {z}_{k} - h ( {\hat{x}}_{k - 1}^{-} , {v}_{k} ) ) \\ {P}_{k} = ( I - {K}_{k} {H}_{k} ) {P}_{k}^{-}\end{matrix}$$

in the above process, φ is a state transition matrix; H is a Jacobian matrix calculated by h function for the state;
T is a sampling time, in which

$$ϕ = I + F \times T$$

where f is a state equation; h is an observation equation; x is a state quantity; u is an input quantity; w and v are a process noise and an observation noise; P is an error covariance matrix; K is a Kalman gain.

In some embodiments, estimating the position coordinate of the positioning tag in Step S24 includes the following steps:
first, the original TDOA value is substituted into the Chan algorithm to calculate and acquire initial positioning coordinates of the UWB positioning tag, and then the residual sum of squares is calculated, in which calculation formula is as follows:

$${R}_{ws} = {\sum }_{i = 2}^{n} [ {R}_{i , 1} - ( \sqrt{{( \hat{x} - {x}_{i} )}^{2} + {( \hat{y} - {y}_{i} )}^{2}} - \sqrt{{{( \hat{x} - {x}_{1} )}^{2} + {( \hat{y} - {y}_{1} )}^{2} ) ]}^{2}}$$

thereafter, the errors are eliminated, the residuals are used to measure the proximity between a set of TDOA values and the corresponding positioning results, when errors of the UWB positioning system mainly comes from Non Line of Sight (NLOS) errors, in response to increasing of an influence of the NLOS errors on a set of measurement values, calculated residual value increases;
therefore, a threshold value is set as R_{ws}≤Δ, and compared with the residual value, the TDOA measurement values with large NLOS errors are eliminated;
when the residual value is greater than the threshold value, it indicates that there is a large deviation between a distance difference between the estimated coordinates calculated by the Chan algorithm and the base station and the TDOA measurement value between the tag and the base station measured by a sensor, when the NLOS errors are considered as main errors of the positioning system, it indicates that the TDOA measurement values contain big NLOS errors, and then a group of corresponding TDOA measurement values are eliminated;
in response to the residual value less than or equal to the threshold value, it is considered that the error of the measurement value is small.

Finally, the position coordinates of the positioning tag are estimated, and the retained TDOA measurement values with small errors are substituted into the standard EKF filtering algorithm, so that the final position coordinate of the surgical instrument is obtained.

In some embodiments, the UWB module unit includes a main control chip, a signal processing chip, a radio frequency power amplifier circuit and a power supply unit, which are electrically connected in sequence, and the radio frequency power amplifier circuit is also electrically connected with a tri-state power buffer.

In some embodiments, electrical connection method between the main control chip and the signal processing chip is bidirectional electrical connection.

In some embodiments, a model of the main control chip is STM32F407, a model of the signal processing chip is DW100, a model of the power supply unit is TPS61240, and a model of the tri-state power buffer is SN74LV1T125.

Compared with the prior art, the present disclosure has the following beneficial effects:
the present disclosure has the advantage that the UWB chip-level wireless positioning module is installed on a surgical instrument. The biggest advantage of the UWB wireless positioning module is that the UWB wireless positioning module has a wide bandwidth, so it has good anti-interference ability. In a positioning and tracking stage, only a base station and modules need to communicate to acquire spatial position of the surgical instruments relative to patients. The wireless positioning technology based on the UWB is very mature at this stage, and there are diverse intraoperative visualization means, and surgical status can be monitored through devices such as mobile phones and tablets. Compared with an existing optical positioning system, the present disclosure has the advantages of good robustness, high cost performance, strong real-time performance and the like.

### BRIEF DESCRIPTION OF THE DRAWINGS

FIG. 1 is a structural block diagram of a UWB module unit according to the present disclosure.

FIG. 2 is a schematic diagram of TDOA three-base-station positioning according to the present disclosure.

FIG. 3 is a schematic diagram of an overall algorithm flow according to the present disclosure.

FIG. 4 is a bandwidth comparison diagram according to the present disclosure.

FIG. 5 is a spectrum comparison diagram according to the present disclosure.

### DETAILED DESCRIPTION OF THE EMBODIMENTS

The technical scheme in the embodiment of the present disclosure will be clearly and completely described with reference to the attached drawings hereinafter. Obviously, the described embodiments are only some of the embodiments of the present disclosure, rather than all of the embodiments. Based on the embodiments in the present disclosure, all other embodiments obtained by those skilled in the art without creative labor belong to the scope of protection of the present disclosure.

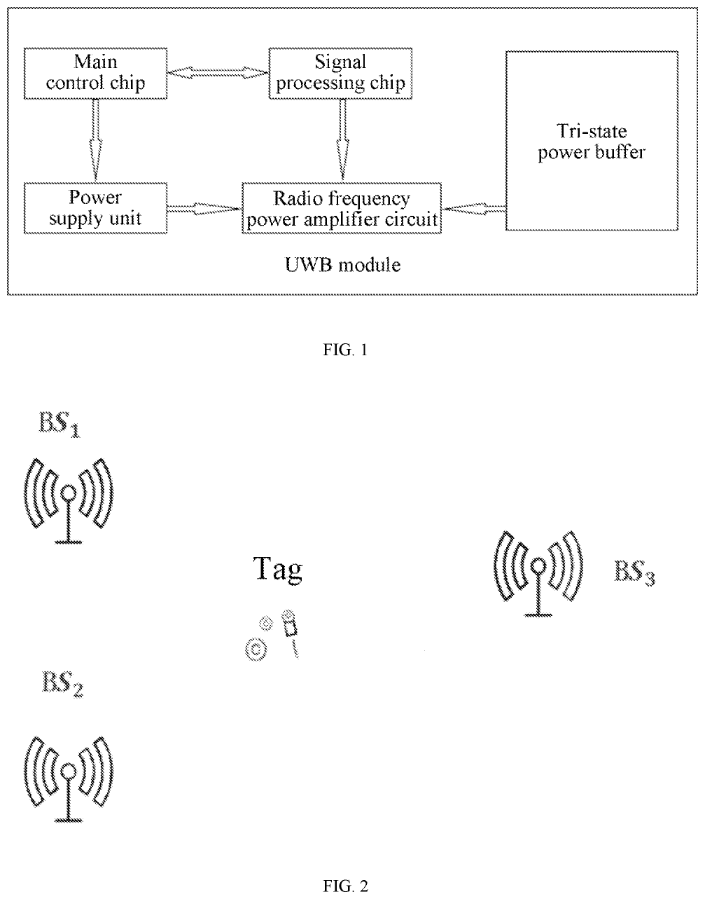

Referring to FIGS. 1 to 5, the present disclosure provides a technical scheme: a chip-level positioning method for orthopedic surgery navigation based on a UWB, including the following steps S1 and S2.

In step S1, a positioning base station is laid and a UWB wireless positioning module is arranged on a surgical instrument, where the positioning base station and the UWB wireless positioning module arranged on the surgical instrument use a same UWB module unit.

In step S2, real-time positioning is performed. Specifically, first, a TDOA value is substituted into a Chan algorithm, to calculate and acquire a preliminary positioning coordinate of a UWB positioning tag. Thereafter, a residual sum of squares is calculated to set a threshold, where the residual sum of squares is used to deal with positioning errors in the UWB wireless positioning module, and TDOA measurement values with errors higher than the threshold are eliminated. And the screened TDOA measurement values that meet requirements are substituted into a standard EKF filtering algorithm, so as to obtain a final position coordinate of the surgical instrument.

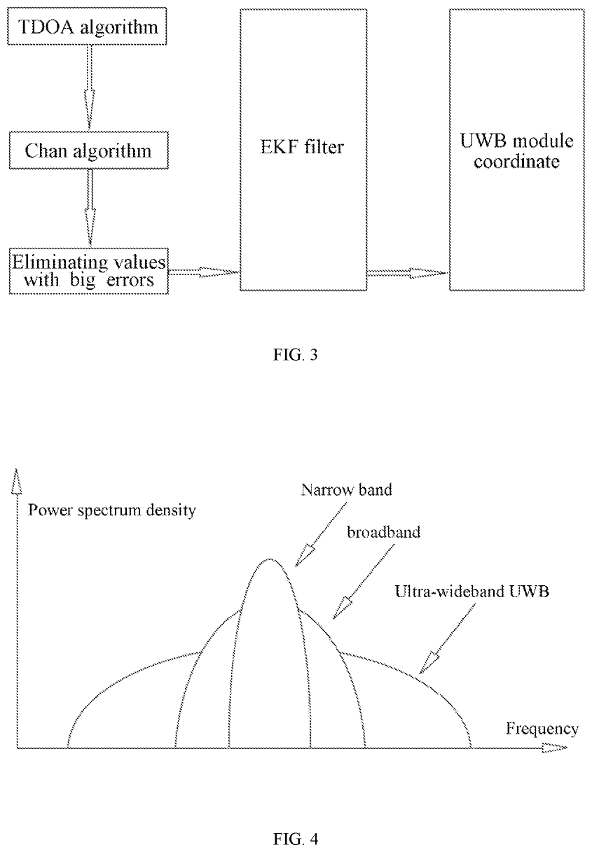

The present disclosure needs to arrange the UWB wireless positioning module on a surgical instrument, and the specific scene layout is arranged according to actual needs. The scheme design mainly consists of two parts: a software design and a hardware design. The specific implementation of the software and the hardware is shown in FIGS. 1 and 3.

In the hardware design part, the positioning base station and the positioning tag use the same UWB module unit. The UWB module unit includes a main control chip, a signal processing chip, a radio frequency power amplifier circuit and a power supply unit which are electrically connected in sequence, and the radio frequency power amplifier circuit is also electrically connected with a tri-state power buffer.

In some embodiments, the electrical connection method between the main control chip and the signal processing chip is bidirectional electrical connection.

In some embodiments, a model of the main control chip is STM32F407, a model of the signal processing chip is DW100, a model of the power supply unit is TPS61240, and a model of the tri-state power buffer is SN74LV1T125.

The DW100 chip of Decawave Company is used as the UWB signal processing chip, which conforms to the wireless standard. In order to enhance the anti-interference ability, the module amplifies the radio frequency signal, uses the TPS61240 chip to supply power to the radio frequency power amplifier circuit, and uses the three-state power buffer SN74LV1T125 to enhance the anti-vibration ability of the power circuit and improve the robustness.

In some embodiments, specific steps in Step S2 include steps S21 to S24 and are described as follows.

In S21, TDOA positioning is performed.
by using a TDOA algorithm, a time difference between moments of two reference base stations receiving broadcast signals sent by a target node is measured, thus a distance difference between distances from the target node to the two reference base stations by multiplying a wave velocity by the time difference. A hyperbolic equation is obtained by taking the two reference base stations as focuses of a curve and taking the distance difference as 2a. One hyperbolic equation is incapable of determining the target node, and at least three base stations are used to solve two hyperbolic equations, and an intersection point of two hyperbolas determined by the two hyperbolic equations is a target node.

In step S22, the Chan algorithm is adopted for positioning based on TDOA technology.

A two-step WLS method is used to locate and calculate a target position, which is suitable for both small-scale and large-scale positioning systems. During a solving process, nonlinear TDOA equations are first processed and converted into linear equations, and then an initial solution is estimated through WLS; and thereafter, the initial solution is calculated through the WLS for a second time to further estimate the coordinate of the positioning tag, where equation conditions used in the first WLS calculation, such as constraints, are the same as those used in the second WLS calculation.

In S23, the EKF algorithm is adopted for positioning based on TDOA technology.

In a UWB positioning system based on TDOA technology, the TDOA equations are nonlinear equations, so an extended Kalman filter is used to solve a nonlinear problem. The idea of the extended Kalman filter is that for a nonlinear system, the system is discretized by means of numerical analysis, then Taylor expansion is carried out in neighborhoods of calculation points, terms above quadratic terms are deleted, and only primary terms are reserved, so that the Kalman filter is applied to the nonlinear system.

In step S24, the position coordinate of the positioning tag is estimated.

The screened TDOA measurement values with small errors is substituted into the standard EKF filtering algorithm, so as to obtain a final position coordinate of the surgical instrument.

In some embodiments, a specific linear equation of TDOA positioning in Step S21 is as follows:
coordinates of various base stations are set to (x₁, y₁), (x₂, y₂) and (x₃, y₃) clockwise from the BS₁, so a tag coordinate to be solved is (x, y), a time for a measure signal traveling from a node to be tested to BS₁ is t₁ (i=2,3), and BS₁ and BSᵢ are taken as focuses, a hyperbolic equation is drawn with D_{i,1}=dᵢ−d₁=2a, and a distance relationship between the node to be tested and the base station i is obtained by a distance formula between two points:

$${D}_{i} = \sqrt{{( {x}_{i} - x )}^{2}} - \sqrt{{( {y}_{i} - y )}^{2}} .$$

A relationship between a distance difference between the distance from the node to be tested to a main positioning base station BS₁ and the distance Dᵢ from the node to be tested to other base stations and the time difference is as follows:

$${D}_{i , 1} = {D}_{i} - {D}_{1} = {ct}_{i , 1} = \sqrt{{( {x}_{i} - x )}^{2} + {( {y}_{i} - y )}^{2}} - \sqrt{{( {x}_{1} - x )}^{2} + {( {y}_{1} - y )}^{2}} ,$$

where c indicates a propagation velocity of an electromagnetic wave emitted by a target in a medium, and more than two base stations besides the main base station are needed to complete the determination of the target node, from which the following hyperbolic nonlinear equations are obtained:

$$\{ \begin{matrix}{D}_{2 , 1} = {D}_{2} - {D}_{1} = \sqrt{{( {x}_{2} - x )}^{2} + {( {y}_{2} - y )}^{2}} - \sqrt{{( {x}_{1} - x )}^{2} + {( {y}_{1} - y )}^{2}} \\ {D}_{3 , 1} = {D}_{3} - {D}_{1} = \sqrt{{( {x}_{3} - x )}^{2} + {( {y}_{3} - y )}^{2}} - \sqrt{{( {x}_{1} - x )}^{2} + {( {y}_{1} - y )}^{2}}\end{matrix} .$$

The x and y are solved using the Chan algorithm to obtain the tag coordinate.

In some embodiments, a specific linear equation of the EKF algorithm based on TDOA technology positioning in Step S23 is described as follows.

Assuming that the nonlinear system is:

$$\{ \begin{matrix}{x}_{k} = f ( {x}_{k - 1 ,} {u}_{k - 1} , {w}_{k - 1} ) \\ {z}_{k} = h ( {x}_{k} , {v}_{k} )\end{matrix} ,$$

a time update equation is:

$$\begin{matrix}{\hat{x}}_{k}^{-} = f ( {\hat{x}}_{k - 1} , {w}_{k - 1} ) \\ {P}_{k}^{-} = ϕ {P}_{k - 1} {ϕ}^{T} + Q\end{matrix} ,$$

an updated measurement equation is:

$${K}_{k} = {P}_{k}^{-} {{H}_{K}^{T} ( {H}_{k} {P}_{k}^{-} {H}_{K}^{T} + R )}^{- 1}$$

$${\hat{x}}_{k} = {\hat{x}}_{k - 1}^{-} + {K}_{k} ( {z}_{k} - h ( {\hat{x}}_{k - 1}^{-} , {ν}_{k} ) ) ,$$

$${P}_{k} = ( I - {K}_{k} {H}_{k} ) {P}_{k}^{-}$$

in the above process, φ is a state transition matrix; H is a Jacobian matrix calculated by h function for the state.
T is a sampling time, in which

$$ϕ = I + F \times T$$

where f is a state equation; h is an observation equation; x is a state quantity; u is an input quantity; w and v are a process noise and an observation noise; P is an error covariance matrix; K is a Kalman gain.

In some embodiments, the estimating the position coordinate of the positioning tag in Step S24 includes the following steps:

First, the original TDOA value is substituted into the Chan algorithm to calculate and acquire initial positioning coordinates of the UWB positioning tag, and then the sum of squared residuals is calculated, in which the calculation formula is as follows:

$${R}_{ws} = {\sum }_{i = 2}^{n} [ {R}_{i , 1} - ( \sqrt{{( \hat{x} - {x}_{i} )}^{2} + {( \hat{y} - {y}_{i} )}^{2}} - \sqrt{{{( \hat{x} - {x}_{1} )}^{2} + {( y - {y}_{1} )}^{2} ) ]}^{2}} .$$

Thereafter, the errors are eliminated, the residuals are used to measure the proximity between a set of TDOA values and the corresponding positioning results, when errors of the UWB positioning system mainly comes from NLOS errors, the greater the influence of the NLOS errors on a set of measurement values, the greater the calculated residual value.

Therefore, a threshold value is set as R_{ws}≤Δ, and compared with the residual value, the TDOA measurement values with large NLOS errors are eliminated.

When the residual value is greater than the threshold value, it indicates that there is a large deviation between a distance difference between the estimated coordinates calculated by the Chan algorithm and the base station and the TDOA measurement value between the tag and the base station measured by a sensor, when the NLOS errors are taken as the main errors of the positioning system, it indicates that the TDOA measurement values contain big NLOS errors, and then a group of corresponding TDOA measurement values are eliminated.

If the residual value is less than or equal to the threshold value, it is considered that the error of the measurement value is small.

Finally, the position coordinates of the positioning tag are estimated, and the screened TDOA measurement values with small errors are substituted into the standard EKF filtering algorithm, so that the final position coordinate of the surgical instrument is obtained.

The wireless positioning technology based on the UWB has the advantages of large system capacity, fast transmission rate, low transmission power and strong real-time performance, which are specifically as follows.

As shown in FIG. 4, the wider the bandwidth, the greater the maximum transmission rate of the system, while the bandwidth of UWB communication is 500 MHz or more, and the transmission rate can reach 1 Gbps or more.

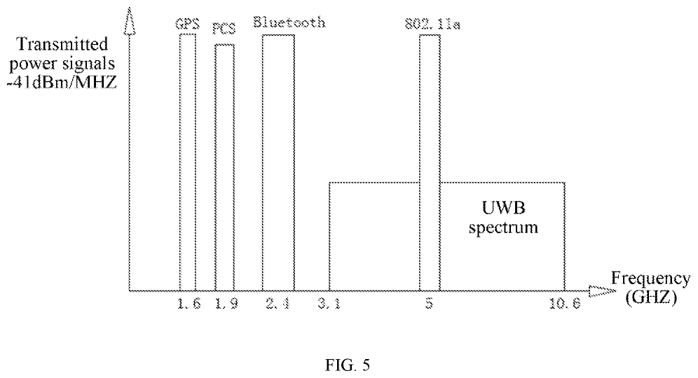

As shown in FIG. 5, the huge bandwidth of the UWB ensures low transmission power. In applications of short-range wireless communication, the UWB signal power transmitted by a transmitter is less than 1 mW, which greatly prolongs the battery life, ensures a long operating time of the system, and has less radiation harm to a human body.

The real-time and accurate measurement of UWB means that the UWB-enabled system can highly determine the precise position of the device and whether the device is stationary or moving.

The present disclosure has the advantage that the UWB chip-level wireless positioning module is installed on a surgical instrument. The biggest advantage of the UWB wireless positioning module is that the UWB wireless positioning module has a wide bandwidth, so it has good anti-interference ability. In a positioning and tracking stage, only a base station and modules need to communicate to acquire spatial position of the surgical instruments relative to patients. The wireless positioning technology based on the UWB is very mature at this stage, and there are diverse intraoperative visualization means, and surgical status can be monitored through devices such as mobile phones and tablets. Compared with an existing optical positioning system, the present disclosure has the advantages of good robustness, high cost performance, strong real-time performance and the like.

Although embodiments of the present disclosure have been shown and described, it will be understood by those skilled in the art that various changes, modifications, substitutions and variations can be made to these embodiments without departing from the principle and the spirit of the present disclosure. The scope of the present disclosure is defined by the appended claims and the equivalents thereof.

What is claimed is:

1. A chip-level positioning method for orthopedic surgery navigation based on an Ultra-wide Bandwidth (UWB), comprising:
S1, laying a positioning base station and arranging a UWB wireless positioning module on a surgical instrument, wherein the positioning base station and the UWB wireless positioning module arranged on the surgical instrument use a same UWB module unit;
S2, real-time positioning: first, substituting an original Time Difference of Arrival (TDOA) value into a Chan algorithm to calculate and acquire a preliminary positioning coordinate of a UWB positioning tag, thereafter, calculating a residual sum of squares to set a threshold, and eliminating TDOA measurement values with errors higher than the threshold, and substituting the screened TDOA measurement values that meet requirements into a standard Extended Kalman Filter (EKF) filtering algorithm, so as to obtain a final position coordinate of the surgical instrument.

2. The chip-level positioning method for orthopedic surgery navigation based on the UWB according to claim 1, wherein Step S2 comprises:
S21, TDOA positioning;
by using a TDOA algorithm, measuring a time difference between moments of two reference base stations receiving broadcast signals sent by a target node, thus calculating a distance difference between distances from the target node to the two reference base stations by multiplying a wave velocity by the time difference, obtaining a hyperbolic equation by taking the two reference base stations as focuses of a curve and taking the distance difference as 2a, wherein one hyperbolic equation is incapable of determining the target node, and at least three base stations are used to solve two hyperbolic equations, and an intersection point of two hyperbolas determined by the two hyperbolic equations is a target node;
S22, the Chan algorithm for positioning based on TDOA technology;
using a two-step Weighted Least Square (WLS) method to locate and calculate a target position, which is suitable for both small-scale and large-scale positioning systems, wherein during the calculating, a nonlinear TDOA equations are first processed and converted into linear equations, and then an initial solution is estimated through WLS; and thereafter, the initial solution is calculated through the WLS for a second time to further estimate coordinate of the positioning tag, wherein equation conditions used in the first WLS calculation are a same as those used in the second WLS calculation;
S23, the EKF algorithm for positioning based on TDOA technology;
in a UWB positioning system based on TDOA technology, considering that the TDOA equations are nonlinear equations, using an EKF to solve a nonlinear problem, wherein the idea of the EKF is that for a nonlinear system, the system is discretized by means of numerical analysis, and Taylor expansion is carried out in a neighborhood of calculation points, terms above quadratic terms are deleted, and only primary terms are reserved, and thus the Kalman filter is applied to the nonlinear system;
S24, estimation of the position coordinate of the positioning tag;
substituting screened TDOA measurement values with small errors into the standard EKF filtering algorithm, so as to obtain a final position coordinate of the surgical instrument.

3. The chip-level positioning method for orthopedic surgery navigation based on the UWB according to claim 2, wherein a specific linear equation of TDOA positioning in Step S21 is as follows:
coordinates of base stations are set to (x₁, y₁), (x₂, y₂) and (x₃, y₃) clockwise from the BS₁, and a tag coordinate to be solved is (x, y), a time for a measure signal traveling from a node to be tested to BS₁ is t₁ (i=2,3), and BS₁ and BSᵢ are deemed as focuses, a hyperbolic equation is drawn with D_{i,1}=dᵢ−d₁=2a, and a distance relationship between the node to be tested and the base station i is obtained by a distance formula between two points:
$${D}_{i} = \sqrt{{( {x}_{i} - x )}^{2}} - \sqrt{{( {y}_{1} - y )}^{2}}$$
a relationship between a distance difference between the distance from the node to be tested to a main positioning base station BS₁ and the distance Dᵢ from the node to be tested to other base stations and the time difference is as follows:
$${D}_{i , 1} = {D}_{i} - {D}_{1} = c {t}_{i , 1} = \sqrt{{( {x}_{i} - x )}^{2} + {( {y}_{i} - y )}^{2}} - \sqrt{{( {x}_{1} - x )}^{2} + {( {y}_{1} - y )}^{2}}$$
c indicates a propagation velocity of an electromagnetic wave emitted by the target in the medium, and more than two base stations besides the main base station are needed to complete the determination of the target node, from which following hyperbolic nonlinear equations are obtained:
$$\{ \begin{matrix}{D}_{2 , 1} = {D}_{2} - {D}_{1} = \sqrt{{( {x}_{2} - x )}^{2} + {( {y}_{2} - y )}^{2}} - \sqrt{{( {x}_{1} - x )}^{2} + {( {y}_{1} - y )}^{2}} \\ {D}_{3 , 1} = {D}_{3} - {D}_{1} = \sqrt{{( {x}_{3} - x )}^{2} + {( {y}_{3} - y )}^{2}} - \sqrt{{( {x}_{1} - x )}^{2} + {( {y}_{1} - y )}^{2}}\end{matrix}$$
x and y are solved using the Chan algorithm to obtain the tag coordinate.

4. The chip-level positioning method for orthopedic surgery navigation based on the UWB according to claim 2, wherein a specific linear equation of the EKF algorithm based on TDOA technology positioning in Step S23 is as follows:
assuming that the nonlinear system is:
$$\{ \begin{matrix}{x}_{k} = f ( {x}_{k - 1} , {u}_{k - 1} , {w}_{k - 1} ) \\ {z}_{k} = h ( {x}_{k} , {v}_{k} )\end{matrix}$$
a time update equation is:
$$\begin{matrix}{\hat{x}}_{k}^{-} = f ( {\hat{x}}_{k - 1} , {u}_{k - 1} , {w}_{k - 1} ) \\ {P}_{k}^{-} = ϕ {P}_{k - 1} {ϕ}^{T} + Q\end{matrix}$$
an updated measurement equation is:
$${K}_{k} = {P}_{k}^{-} {{H}_{K}^{T} ( {H}_{k} {P}_{k}^{-} {H}_{K}^{T} + R )}^{- 1}$$
$${\hat{x}}_{k} = {\hat{x}}_{k - 1}^{-} + {K}_{k} ( {z}_{k} - h ( {\hat{x}}_{k - 1}^{-} , {ν}_{k} ) )$$
$${P}_{k} = ( I - {K}_{k} {H}_{k} ) {P}_{k}^{-}$$
in the above process, φ is a state transition matrix; H is a Jacobian matrix calculated by h function for the state;
T is a sampling time, in which
$$ϕ = I + F \times T$$
where f is a state equation; h is an observation equation; x is a state quantity; u is an input quantity; w and v are a process noise and an observation noise; P is an error covariance matrix; K is a Kalman gain.

5. The chip-level positioning method for orthopedic surgery navigation based on the UWB according to claim 2, wherein estimating the position coordinate of the positioning tag in Step S24 comprises:
first, the original TDOA value is substituted into the Chan algorithm to calculate and acquire initial positioning coordinates of the UWB positioning tag, and then the residual sum of squares is calculated, in which calculation formula is as follows:
$${R}_{w s} = {\sum }_{i = 2}^{n} [ {R}_{i , 1} - ( \sqrt{{( \hat{x} - {x}_{i} )}^{2} + {( \hat{y} - {y}_{i} )}^{2}} - \sqrt{{{( \hat{x} - {x}_{1} )}^{2} + {( \hat{y} - {y}_{1} )}^{2} ) ]}^{2}}$$
thereafter, the errors are eliminated, the residuals are used to measure proximity between a set of TDOA values and corresponding positioning results, when errors of the UWB positioning system mainly comes from Non Line of Sight (NLOS) errors, in response to increasing of an influence of the NLOS errors on a set of measurement values, calculated residual value increases;
therefore, a threshold value is set as R_{ws}≤Δ, and compared with the residual value;
in response to the residual value being greater than the threshold value, corresponding TDOA measurement values are eliminated;
in response to the residual value being less than or equal to the threshold value, corresponding measurement values are retained;
finally, the position coordinates of the positioning tag are estimated, and the retained TDOA measurement values are substituted into the standard EKF filtering algorithm, so that the final position coordinate of the surgical instrument is obtained.

6. The chip-level positioning method for orthopedic surgery navigation based on the UWB according to claim 1, wherein the UWB module unit comprises a main control chip, a signal processing chip, a radio frequency power amplifier circuit and a power supply unit which are electrically connected in sequence, and the radio frequency power amplifier circuit is also electrically connected with a tri-state power buffer.

7. The chip-level positioning method for orthopedic surgery navigation based on the UWB according to claim 6, wherein electrical connection method between the main control chip and the signal processing chip is bidirectional electrical connection.

8. The chip-level positioning method for orthopedic surgery navigation based on the UWB according to claim 6, wherein a model of the main control chip is STM32F407, a model of the signal processing chip is DW100, a model of the power supply unit is TPS61240, and a model of the tri-state power buffer is SN74LV1T125.
